(19) United States
(12) Plant Patent Application Publication
(10) Pub. No.: US 2002/0002726 P1
(43) Pub. Date: Jan. 3, 2002

(54) EUPHORBIA CYATHOPHORA PLANT NAMED 'YOKOI'S WHITE'

(76) Inventor: MASATO YOKOI, KAWAGUCHI CITY (JP)

Correspondence Address: CHRISTIE PARKER & HALE LLP, P O BOX 7068, PASADENA, CA, 911097068

(21) Appl. No.: 09/311,102

(22) Filed: May 13, 1999

(51) Int. Cl. A01H 5/00

(52) U.S. Cl. PLT/302

(57) ABSTRACT

A new plant variety of Euphorbia cyathophora having cream yellow-margined variegated foliage.

### BACKGROUND OF THE INVENTION

The present invention relates to a new and distinct variety of Euphorbia cyathophora J. Murray which was discovered as a branch sport in a controlled planting of Euphorbia cyathophora in Okinawa, Japan. The varietal denomination of the new variety is ‘Yokoi's White’.

### SUMMARY OF THE INVENTION

The genus Euphorbia is included in the family Euphorbiaceae that comprises about 283 genera of monoecious and dioecious herbs, shrubs or trees, many succulent, with milky white latex. Euphorbia comprises over 2,000 species of which some possess desirable ornamental characteristics. Euphorbia cyathophora is a member of the small subgenus Poinsettia. This subgenus contains plants that are non-succulent, New World herbs and shrubs.

Characteristics of this subgenus are reduced number of nectaries, normally one or two. Of Euphorbia cyathophora flowers cyathia are in terminal clusters, glands 1 or 2, more or less 2-lipped, opening between lips narrowly oblong.

Euphorbia cyathophora is widespread and native throughout much of tropical and subtropical America. It has been introduced and naturalized throughout many parts of the world, including Japan. It generally has linear to ovate or pandurate, dull green leaves. The upper leaves and bracts are green or red based. This plant is often misidentified, cultivated and sold as Euphorbia heterophylla. Synonyms also include Euphorbia heterophylla L. variety cyathophora (Murray) Grisebach; and Poinsettia cyathophora (Murray) Klotzch & Garcke.

### SUMMARY OF THE INVENTION

Among the characteristics of the new variety which distinguish it from its parent, Euphorbia cyathophora and other varieties are its cream yellow colored-margined variegated foliage and distinctive red bract.

Asexual reproduction of the new variety by stem cuttings performed in Okinawa Prefecture, Japan; Lewisberry, Pa.; and Fulshear, Tex. have confirmed that the distinctive characteristics of the new variety are stable and transmitted to succeeding generations.

### COMPARISON WITH PARENT

Among the differences, the new variety differs from its parent in having cream yellow leaf margins about 1 to 5 mm wide, instead of entirely green leaves.

### BRIEF DESCRIPTION OF ILLUSTRATIONS

The accompanying illustrations show specimens of the new cultivar in photo illustrations as true to color as is reasonably possible to make in an illustration of this character.

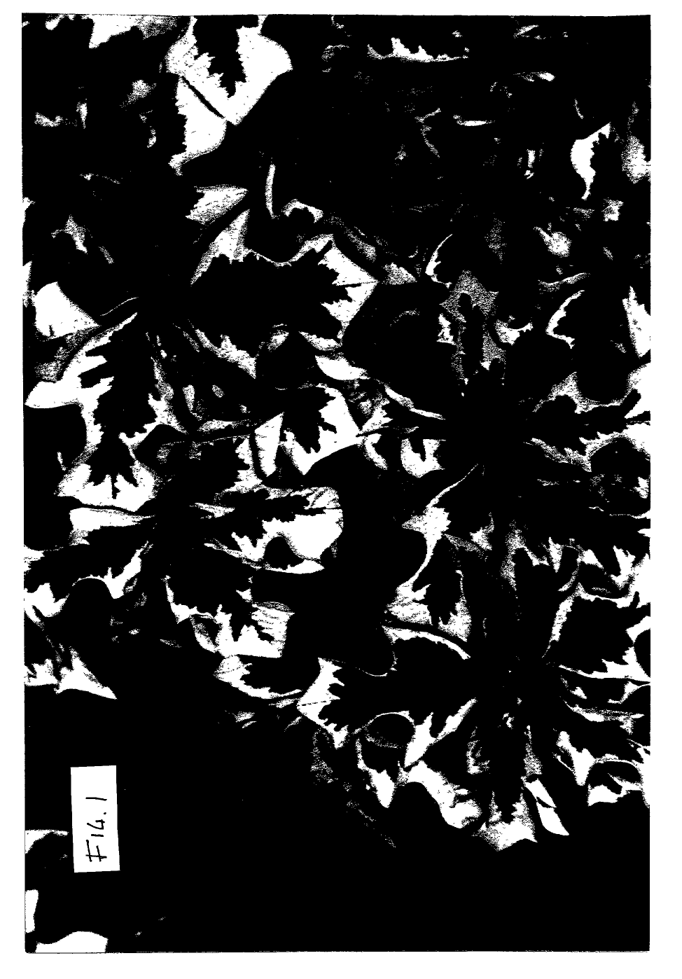

FIG. 1 illustrates the foliage of the new variety and shows the distinctive pattern of variegation; and

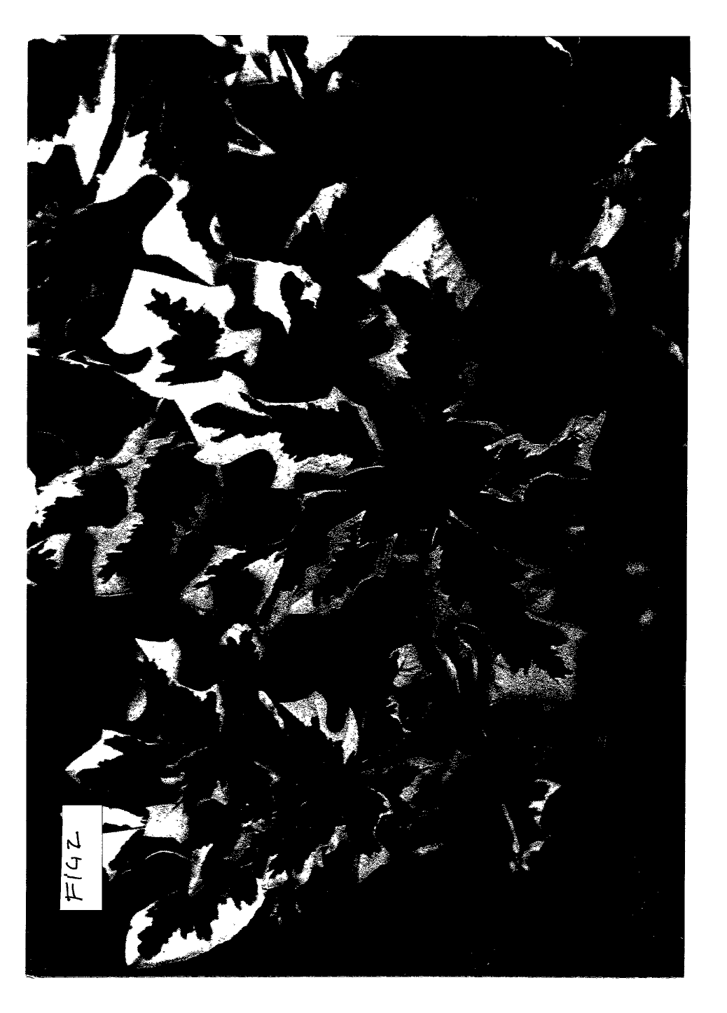

FIG. 2 illustrates the foliage of the new variety with the distinctive red bract and variegated foliage.

### DESCRIPTION OF THE NEW VARIETY

Euphorbia cyathophora ‘Yokoi's White’ has not been observed under all possible environmental, cultural, and light conditions. The following observations and descriptions are of plants grown in containers at Lewisberry, Pa., and Fulshear, Tex. In this description, color references are to The Royal Horticultural Society Colour Chart (1966) and terminology used in the color descriptions herein refers to plate numbers in this color chart. Phenotypic expression may vary with light intensity, cultural, and environmental conditions.

Classification:

Botanical.—Euphorbia cyathophora ‘Yokoi's White’.

Parentage.—Branch sport of the variety Euphorbia cyathophora.

Propagation.—By vegetative cuttings and other known asexual reproduction techniques.

Plant:

Size.—Mature plant can grow to about 50-70 cm at maturity.

Habit.—Upright growing perennial or annual herb.

Leaves.—Most often pandurate, occasionally simple, entire; most often alternate to whorled; occasionally opposite; 60-80 mm long, hairless above the foliage.

Stems.—Near 146A to 146C.

Leaf margin.—Most often pandurate, occasionally entire to dentate, sometimes undulate.

Central sections of foliage.—Near 137B with occasional near 148C on the outer margins of the green central areas; occasional leaf base near 35B to 35D.

Leaf undersurface.—Central near 148B to 148D; margin near 11B to 11D.

Leaf size.—Average about 1.5 to 3.5 cm wide; about 4 to 6 cm long.

Leaf shape.—Occasionally simple, most often pandurate; entire to dentate, sometimes undulated.

Texture and substance.—Firm, undulated, top nearly hairless, underside slightly hirsute.

Leaf apex.—Occasionally acute; usually pandurate.

Petioles.—About 1 cm long, slightly hairy.

Hardiness.—USDA zone 9A (25° to 20° F.) AHS Heat Zone 1.

Bracts.—Reddish bracts occasionally fuse to the foliage; often the bracts are separated from the foliage; bracts are up to about 30 mm long and about 10-20 mm wide; center bract, upper surface is near 34A to 35D; occasionally center bract near 155A.

Bracts undersides.—No reddish coloration.

Inflorescence:

Bloom period.—During growing season.

Flower form.—Cyathium irregular clustered at the tops of 2-forked stems and branches.

Flower arrangement.—Terminal cluster.

Color.—Near 8B.

Fragrance.—None.

Cyathia.—About 2-2.5 mm long, or stalked about 1-3 mm long.

Nectaries.—1, occasionally 2, appearing 2-lipped.

Capsule.—Rounded three-lobed, about 3-4.5 mm long.

Seed.—Dark brown; about 2.5-3 mm long; ovoid or rounded.

I claim:

1. A new and distinct Euphorbia cyathophora plant of the variety substantially as shown and described.
